(19) United States
(12) Patent Application Publication
(10) Pub. No.: US 2021/0331991 A1
(43) Pub. Date: Oct. 28, 2021

(54) ELECTROLUMINESCENT MATERIAL

(71) Applicant: Wuhan China Star Optoelectronics Semiconductor Display Technology Go., Ltd., Wuhan (CN)

(72) Inventor: Yamin WANG, Wuhan (CN)

(21) Appl. No.: 16/488,612

(22) PCT Filed: May 23, 2019
(86) PCT No.: PCT/CN2019/088068

§ 371 Date: Aug. 26, 2019

(30) Foreign Application Priority Data
Apr. 26, 2018 (CN) 201910342201.2

(51) Int. Cl. C07C 15/28; C09K 11/06

(52) U.S. Cl. CPC C07C 15/28; C09K 2211/1011; C09K 11/06

(57) ABSTRACT

An electroluminescent material is provided. 9,9′-bianthracene is used as a homodivalent electron group. The homodivalent electron group in the final compound has mainly functions of absorption and emission and also can control the size of the final molecule. Therefore, a homodivalent system is achieved. Specifically, an electroluminescent material having a wide bandgap, high fluorescence quantum yield, and good thermal stability is prepared by a reaction of 9,9′-bianthryl derivative and 1-bromo-3,5-biphenyl, 9,9′-bianthryl derivative and 1-bromobenzene-3,5-biphenyl, and 9,9′-bianthryl derivative and a mixture of 1-bromo-3,5-biphenyl and 1-bromobenzene-3,5-biphenyl, respectively. Therefore, luminescent efficiency of the electroluminescent material is improved.

### BACKGROUND OF INVENTION

### Field of Invention

The present invention relates to a field of display technology, and more particularly, to an electroluminescent material.

### Description of Prior Art

Organic light emitting diode (OLED) devices are also known as organic electroluminescent display devices and organic light emitting semiconductors. Each OLED device is sandwich structure, which includes transparent indium tin oxide (ITO) film having semiconductor characteristics connected to positive electrode and metal cathode. The entire structural layer includes hole transport layer (HTL), an electroluminescent layer (EL), and an electron transport layer (ETL). When the power is supplied to an appropriate voltage, positive holes and negative electrons are combined in the electroluminescent layer. Under the action of Coulomb force, the electrons and the holes are combined at a certain probability to form excitons (electron-hole pairs). Specifically, excitons refer to electronic excited states that are unstable in the normal environment. The excited excitons are recombined and transfer energy to the electroluminescent material, and the electroluminescent material transits from a ground state to an excited state. The excited state energy generates photons through the radiation relaxation process, and it releases light energy and generates light. Red, green and blue, which are three primary colors, form the basic color according to different formulas.

First, the OLED display devices are self-luminous and unlike the thin film transistor-liquid crystal displays (TFT-LCDs), which require a backlight, so the OLED display devices have high visibility and brightness. Secondly, the OLED display devices have the advantages of low voltage demand, high energy saving efficiency, quick response times, light weight, thin thickness, simple structure, low cost, wide viewing angle, high contrast, low power consumption and high reaction speed. Therefore, the OLED display devices have become one of the most important display technologies and are gradually replacing TFT-LCD, and they are expected to become the next generation main display technology after LCDs.

Organic electroluminescent materials began in 1990, and poly p-phenylenevinylene (PPV) organic light emitting diodes were developed by J. Burroughes and Richard Friend of the University of Cambridge, England. Since then, people have generally used red, green, and blue luminescent materials to achieve full color display. Among the three primary colors, red and green diodes are close to the requirements of practical applications, but the blue luminescent material has a wide band gap and a low highest occupied molecular orbit (HOMO) level, resulting in high charge injection barriers in the devices. Meanwhile, the blue OLED devices are relatively inferior to green and red in terms of electroluminescence (EL) efficiency and device lifetime due to problems such as high emission energy, instability, and emission color impurity caused by easy energy transfer.

Luminescent materials can be classified into fluorescent materials and phosphorescent materials. In the case of blue luminescent materials having a wide band gap, the fluorescent materials exhibit higher efficiency, a wider color gamut, and a longer lifetime than the phosphorescent materials. The phosphorescent blue luminescent materials have a lower triplet level (T1) than the fluorescent material having the singlet state (S1), and it is difficult to develop phosphorescent blue luminescent materials due to the limitation of molecular structure. Since current electroluminescent materials have low luminous efficiency, it is necessary to develop a novel electroluminescent material.

### SUMMARY OF INVENTION

An electroluminescent material is provided to solve the problem of low luminous efficiency of conventional electroluminescent materials.

An electroluminescent material prepared by a raw material, and the raw material includes a first compound and a second compound. A homodivalent electron group of the first compound includes one of an anthracene group, a pyrene group, a carbazole group, or a fluorene group. The second compound includes one of phenyl-substituted biphenyl group and a derivative thereof, phenyl-substituted binaphthalene and a derivative thereof, and phenyl-substituted bianthryl and a derivative thereof.

In one embodiment, the anthracene group is 9,9′-bianthracene.

In one embodiment, the 9,9′-bianthracene has a chemical formula as follows:

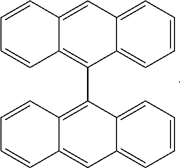 [SMILES: c1ccc2c(-c3c4ccccc4cc4ccccc34)c3ccccc3cc2c1]

In one embodiment, the electroluminescent material includes 10,10′-di-triphenyl-9,9′-bianthryl derivative, which has a chemical formula as follows:

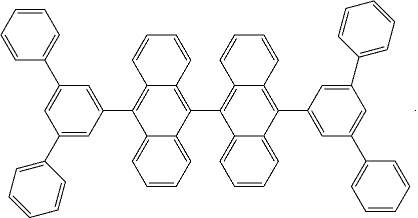 [SMILES: c1ccc(-c2cc(-c3ccccc3)cc(-c3c4ccccc4c(-c4c5ccccc5c(-c5cc(-c6ccccc6)cc(-c6ccccc6)c5)c5ccccc45)c4ccccc34)c2)cc1]

In one embodiment, the first compound is 9,9′-bianthryl derivative, and the second compound is 1-bromo-3,5-biphenyl, and the 9,9′-bianthryl derivative has a chemical formula as follows:

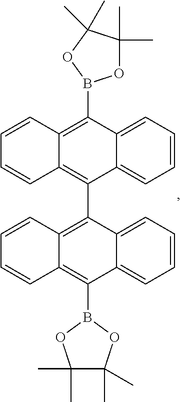 [SMILES: CC1(C)OB(c2c3ccccc3c(-c3c4ccccc4c(B4OC(C)(C)C(C)(C)O4)c4ccccc34)c3ccccc23)OC1(C)C]

and the 1-bromo-3,5-biphenyl has a chemical formula as follows:

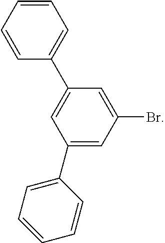 [SMILES: Brc1cc(-c2ccccc2)cc(-c2ccccc2)c1]

In one embodiment, the electroluminescent material includes 10,10′-di-tetraphenyl-9,9′-bianthryl derivative has a chemical formula as follows:

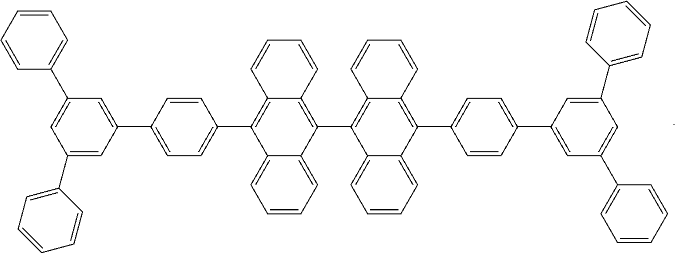 [SMILES: c1ccc(-c2cc(-c3ccccc3)cc(-c3ccc(-c4c5ccccc5c(-c5c6ccccc6c(-c6ccc(-c7cc(-c8ccccc8)cc(-c8ccccc8)c7)cc6)c6ccccc56)c5ccccc45)cc3)c2)cc1]

In one embodiment, the first compound is 9,9′-bianthryl derivative, and the second compound is 1-bromo-3,5-biphenyl, and the 9,9′-bianthryl derivative has a chemical formula as follows:

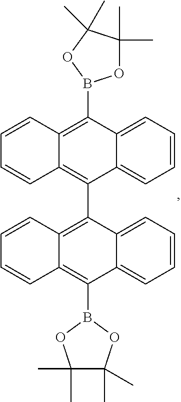 [SMILES: CC1(C)OB(c2c3ccccc3c(-c3c4ccccc4c(B4OC(C)(C)C(C)(C)O4)c4ccccc34)c3ccccc23)OC1(C)C]

and the 1-bromobenzene-3,5-biphenyl has a chemical formula as follows:

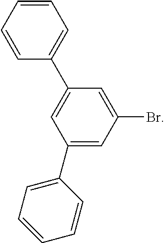 [SMILES: Brc1cc(-c2ccccc2)cc(-c2ccccc2)c1]

In one embodiment, the electroluminescent material includes 10-triphenyl, 10′-tetraphenylenyl-9,9′-bianthryl derivative, and the 10-triphenyl, 10′-tetraphenylenyl-9,9′-bianthryl derivative has a chemical formula as follows:

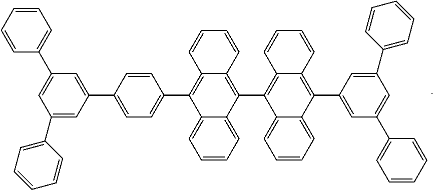 [SMILES: c1ccc(-c2cc(-c3ccccc3)cc(-c3ccc(-c4c5ccccc5c(-c5c6ccccc6c(-c6cc(-c7ccccc7)cc(-c7ccccc7)c6)c6ccccc56)c5ccccc45)cc3)c2)cc1]

In one embodiment, the first compound is 9,9′-bianthryl derivative, and the second compound is a mixture of 1-bromo-3,5-biphenyl and 1-bromobenzene-3,5-biphenyl.

In one embodiment, a ratio of a weight percentage of the 1-bromo-3,5-biphenyl to the 1-bromobenzene-3,5-biphenyl ranges from 0.8 to 1.2.

An electroluminescent material is provided. 9,9′-bianthracene is used as a homodivalent electron group. The homodivalent electron group in the final compound has mainly functions of absorption and emission and also can control the size of the final molecule. Therefore, a homodivalent system is achieved. Specifically, an electroluminescent material having a wide bandgap, high fluorescence quantum yield, and good thermal stability is prepared by a reaction of 9,9′-bianthryl derivative and 1-bromo-3,5-biphenyl, 9,9′-bianthryl derivative and 1-bromobenzene-3,5-biphenyl, and 9,9′-bianthryl derivative and a mixture of 1-bromo-3,5-biphenyl and 1-bromobenzene-3,5-biphenyl, respectively. Therefore, luminescent efficiency of the electroluminescent material is improved.

### BRIEF DESCRIPTION OF DRAWINGS

In order to more clearly illustrate the technical solutions in the embodiments of the present invention, the drawings used in the description of the embodiments will be briefly described below. It is obvious that the drawings in the following description are only some embodiments of the present invention. Other drawings can also be obtained from those skilled persons in the art based on these drawings without paying any creative effort.

FIG. 1 is a luminescence spectrum of electroluminescent materials according to embodiment of the present invention.

### DETAILED DESCRIPTION OF PREFERRED EMBODIMENTS

The preferred embodiments of the present invention are described in detail below with reference to the accompanying drawings. Those skilled persons in the art will easily understand how to implement the invention. The invention can be implemented by the embodiments, so that the technical content of the disclosure will be clear, so that those skilled persons in the art will understand how to implement the invention. The present invention may be accomplished in many different embodiments, and the scope of the invention is not limited to the embodiments described herein.

Directional terms mentioned in this application, such as “up,” “down,” “forward,” “backward,” “left,” “right,” “inside,” “outside,” “side,” etc., are merely indicated the direction of the drawings. Therefore, the directional terms are used for illustrating and understanding of the application rather than limiting thereof.

In the drawings, identical components are marked with the same reference numerals, and structural or components having similar functions are marked with similar reference numerals. Moreover, the size and thickness of each component shown in the drawings are arbitrarily shown for understanding and describing, and the invention does not limit the size and thickness of each component.

When a component is described as “on” another component, the component can be disposed directly on the other component. Also, one component is disposed on an intermediate component, and the intermediate component is disposed on another component. When a component is described as “installed” or “connected” to another component, it can be understood as directly “installed” or “connected” to another component.

In a first embodiment, an electroluminescent material is prepared by a raw material, and the raw material includes a first compound and a second compound. A homodivalent electron group of the first compound includes one of an anthracene group, a pyrene group, a carbazole group, or a fluorene group. The second compound includes one of phenyl-substituted biphenyl group and a derivative thereof, phenyl-substituted binaphthalene and a derivative thereof, and phenyl-substituted bianthryl and a derivative thereof. Specifically, the anthracene group is 9,9′-bianthracene has a chemical formula as follows:

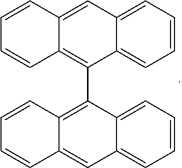 [SMILES: c1ccc2c(-c3c4ccccc4cc4ccccc34)c3ccccc3cc2c1]

The 10,10′-di-triphenyl-9,9′-bianthryl derivative has a chemical formula as follows:

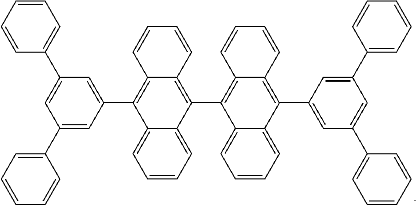 [SMILES: c1ccc(-c2cc(-c3ccccc3)cc(-c3c4ccccc4c(-c4c5ccccc5c(-c5cc(-c6ccccc6)cc(-c6ccccc6)c5)c5ccccc45)c4ccccc34)c2)cc1]

The first compound is 9,9′-bianthryl derivative, and the second compound is 1-bromo-3,5-biphenyl, and the 9,9′-bianthryl derivative has a chemical formula as follows:

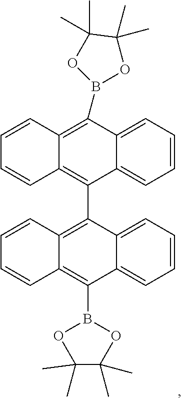 [SMILES: CC1(C)OB(c2c3ccccc3c(-c3c4ccccc4c(B4OC(C)(C)C(C)(C)O4)c4ccccc34)c3ccccc23)OC1(C)C]

and the 1-bromo-3,5-biphenyl has a chemical formula as follows:

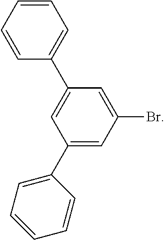 [SMILES: Brc1cc(-c2ccccc2)cc(-c2ccccc2)c1]

The specific preparation steps are described. 4-30 mmol 9,9′-bianthryl derivative, 0.15-0.6 mmol Pd catalyst, and 0.3-1.9 mmol of tricyclohexyl phosphine are added to anhydrous toluene and anhydrous ethanol solution. 1-bromo-3,5-biphenyl is added to a 100 mL beaker, then ethanolamine is slowly added to the beaker by using a pipette and dissolved in the beaker. Next, 15-30 mL ethanolamine including dissolved 1-bromo-3,5-biphenyl is added to the above reaction mixture by using a syringe, and the reaction mixture is refluxed under argon for 4 hours. After the reaction is completed, the reaction mixture is extracted with chloroform and water. The organic layer is dried over anhydrous MgSO₄ and filtered, and then the solution is evaporated. Finally, a product is isolated by silica gel column chromatography to obtain 10,10′-di-triphenyl-9,9′-bianthryl derivative.

In the first embodiment, 9,9′-bianthracene is used as a homodivalent electron group. The homodivalent electron group in the final compound has mainly functions of absorption and emission and also can control the size of the final molecule. Therefore, a homodivalent system is achieved. Specifically, an electroluminescent material having a wide bandgap, high fluorescence quantum yield, and good thermal stability is prepared by a reaction of 9,9′-bianthryl derivative and 1-bromo-3,5-biphenyl. Therefore, luminescent efficiency of the electroluminescent material is improved.

In a second embodiment, the difference between the second embodiment and the first embodiment is described below, and the others are not be described herein.

An electroluminescent material is 10,10′-di-tetraphenyl-9,9′-bianthryl derivative, which has a chemical formula as follows:

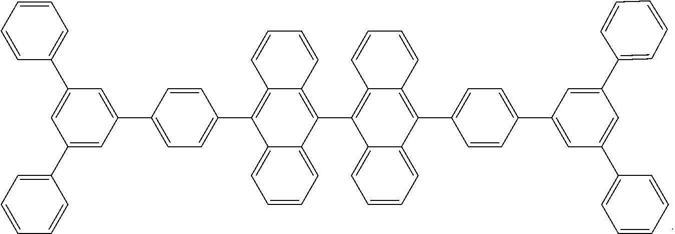 [SMILES: c1ccc(-c2cc(-c3ccccc3)cc(-c3ccc(-c4c5ccccc5c(-c5c6ccccc6c(-c6ccc(-c7cc(-c8ccccc8)cc(-c8ccccc8)c7)cc6)c6ccccc56)c5ccccc45)cc3)c2)cc1]

The first compound is 9,9′-bianthryl derivative, and the second compound is 1-bromo-3,5-biphenyl, and the 9,9′-bianthryl derivative has a chemical formula as follows:

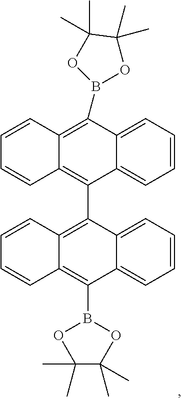 [SMILES: CC1(C)OB(c2c3ccccc3c(-c3c4ccccc4c(B4OC(C)(C)C(C)(C)O4)c4ccccc34)c3ccccc23)OC1(C)C]

and the 1-bromobenzene-3,5-biphenyl has a chemical formula as follows:

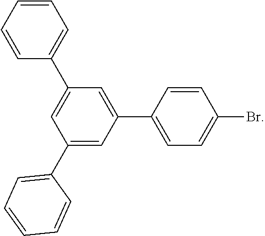 [SMILES: Brc1ccc(-c2cc(-c3ccccc3)cc(-c3ccccc3)c2)cc1]

The specific preparation steps are described. 4-30 mmol 9,9′-bianthryl derivative, 0.15-0.6 mmol Pd catalyst, and 0.3-1.9 mmol of tricyclohexyl phosphine are added to anhydrous toluene and anhydrous ethanol solution. 1-bromobenzene-3,5-biphenyl is added to a 100 mL beaker, then ethanolamine is slowly added to the beaker by using a pipette and dissolved in the beaker. Next, 15-30 mL ethanolamine including dissolved 1-bromobenzene-3,5-biphenyl is added to the above reaction mixture by using a syringe, and the reaction mixture is refluxed under argon for 4 hours. After the reaction is completed, the reaction mixture is extracted with chloroform and water. The organic layer is dried over anhydrous MgSO₄ and filtered, and then the solution is evaporated. Finally, a product is isolated by silica gel column chromatography to obtain 10,10′-di-tetraphenyl-9,9′-bianthryl derivative.

In the second embodiment, 9,9′-bianthracene is used as a homodivalent electron group. The homodivalent electron group in the final compound has mainly functions of absorption and emission and also can control the size of the final molecule. Therefore, a homodivalent system is achieved. Specifically, an electroluminescent material having a wide bandgap, high fluorescence quantum yield, and good thermal stability is prepared by a reaction of 9,9′-bianthryl derivative and 1-bromobenzene-3,5-biphenyl. Therefore, luminescent efficiency of the electroluminescent material is improved.

In a third embodiment, the difference between the third embodiment and the first embodiment is described below, and the others are not be described herein.

An electroluminescent material is 10-triphenyl, 10′-tetraphenylenyl-9,9′-bianthryl derivative, which has a chemical formula as follows:

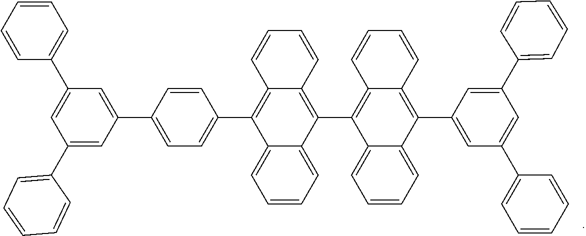 [SMILES: c1ccc(-c2cc(-c3ccccc3)cc(-c3ccc(-c4c5ccccc5c(-c5c6ccccc6c(-c6cc(-c7ccccc7)cc(-c7ccccc7)c6)c6ccccc56)c5ccccc45)cc3)c2)cc1]

The first compound is 9,9′-bianthryl derivative, and the second compound is a mixture of 1-bromo-3,5-biphenyl and 1-bromobenzene-3,5-biphenyl.

The specific preparation steps are described. 4-30 mmol 9,9′-bianthryl derivative, 0.15-0.6 mmol Pd catalyst, and 0.3-1.9 mmol of tricyclohexyl phosphine are added to anhydrous toluene and anhydrous ethanol solution. 1-bromo-3,5-biphenyl and 1-bromobenzene-3,5-biphenyl are added to a 100 mL beaker, then ethanolamine is slowly added to the beaker by using a pipette and dissolved in the beaker. Next, 15-30 mL ethanolamine including dissolved 1-bromo-3,5-biphenyl and 1-bromobenzene-3,5-biphenyl is added to the above reaction mixture by using a syringe, and the reaction mixture is refluxed under argon for 4 hours. After the reaction is completed, the reaction mixture is extracted with chloroform and water. The organic layer is dried over anhydrous MgSO₄ and filtered, and then the solution is evaporated. Finally, a product is isolated by silica gel column chromatography to obtain 10-triphenyl, 10′-tetraphenylenyl-9,9′-bianthryl derivative.

A ratio of a weight percentage of the 1-bromo-3,5-biphenyl to the 1-bromobenzene-3,5-biphenyl ranges from 0.8 to 1.2.

In the first embodiment, 9,9′-bianthracene is used as a homodivalent electron group. The homodivalent electron group in the final compound has mainly functions of absorption and emission and also can control the size of the final molecule. Therefore, a homodivalent system is achieved. Specifically, an electroluminescent material having a wide bandgap, high fluorescence quantum yield, and good thermal stability is prepared by a reaction of 9,9′-bianthryl derivative and a mixture of 1-bromo-3,5-biphenyl and 1-bromobenzene-3,5-biphenyl. Therefore, luminescent efficiency of the electroluminescent material is improved.

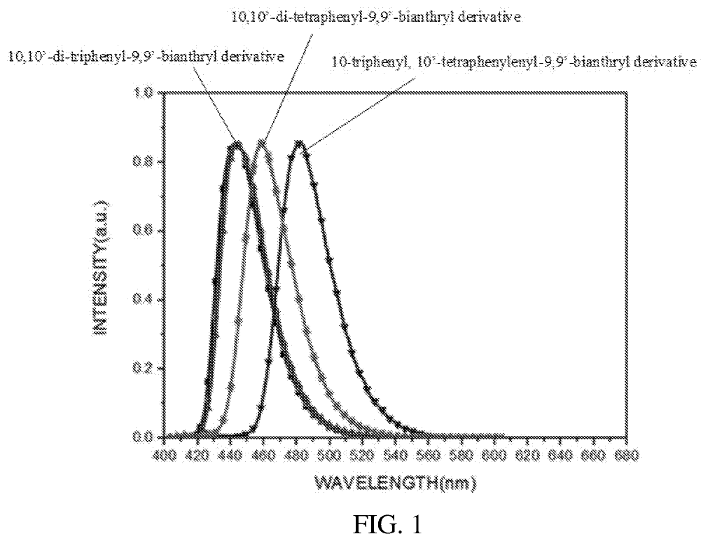

As shown in FIG. 1, three electroluminescent materials according to the three embodiments have a maximum emission peak at 435-480 nm, which are high-efficiency blue luminescent materials. Due to the specific structure of the compound, a blue shift of the electroluminescent material is occurred in the spectrum with an increasing number of benzene rings. However, 9,9′-bianthracene used as a homodivalent electron group limits the size of the molecule, which acts as a core in the molecule. Therefore, molecule size cannot be increased without limit, which only causes the material to fail to emit blue light. At the same time, it will inhibit the π-π accumulation of adjacent molecules, because of its relatively large steric hindrance.

In the above, the present application has been described in the above preferred embodiments, but the preferred embodiments are not intended to limit the scope of the invention, and a person skilled in the art may make various modifications without departing from the spirit and scope of the application. The scope of the present application is determined by claims.

What is claimed is:

1. An electroluminescent material prepared by a raw material, wherein the raw material comprises:
a first compound, wherein a homodivalent electron group of the first compound comprises one of an anthracene group, a pyrene group, a carbazole group, or a fluorene group; and
a second compound, wherein the second compound comprises one of phenyl-substituted biphenyl group and a derivative thereof, phenyl-substituted binaphthalene and a derivative thereof, and phenyl-substituted bianthryl and a derivative thereof.

2. The electroluminescent material prepared by and raw material according to claim 1, wherein the anthracene group is 9,9′-bianthracene.

3. The electroluminescent material prepared by and raw material according to claim 2, wherein the 9,9′-bianthracene has a chemical formula as follows:

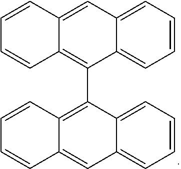 [SMILES: c1ccc2c(-c3c4ccccc4cc4ccccc34)c3ccccc3cc2c1]

4. The electroluminescent material prepared by and raw material according to claim 1, further comprising 10,10′-di-triphenyl-9,9′-bianthryl derivative, wherein the 10,10′-di-triphenyl-9,9′-bianthryl derivative has a chemical formula as follows:

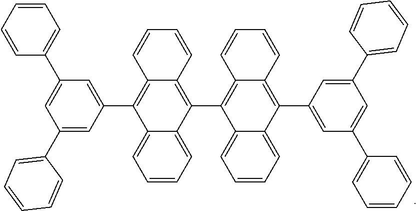 [SMILES: c1ccc(-c2cc(-c3ccccc3)cc(-c3c4ccccc4c(-c4c5ccccc5c(-c5cc(-c6ccccc6)cc(-c6ccccc6)c5)c5ccccc45)c4ccccc34)c2)cc1]

5. The electroluminescent material prepared by and raw material according to claim 4, wherein the first compound is 9,9′-bianthryl derivative, and the second compound is 1-bromo-3,5-biphenyl, and the 9,9′-bianthryl derivative has a chemical formula as follows: 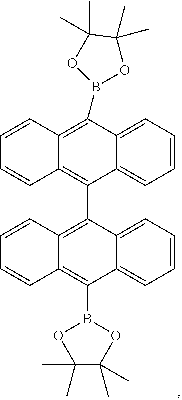 [SMILES: CC1(C)OB(c2c3ccccc3c(-c3c4ccccc4c(B4OC(C)(C)C(C)(C)O4)c4ccccc34)c3ccccc23)OC1(C)C]
and the 1-bromo-3,5-biphenyl has a chemical formula as follows:

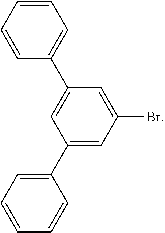 [SMILES: Brc1cc(-c2ccccc2)cc(-c2ccccc2)c1]

6. The electroluminescent material prepared by and raw material according to claim 1, further comprising 10,10′-di-tetraphenyl-9,9′-bianthryl derivative has a chemical formula as follows:

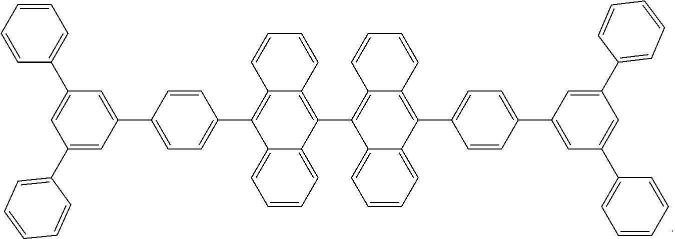 [SMILES: c1ccc(-c2cc(-c3ccccc3)cc(-c3ccc(-c4c5ccccc5c(-c5c6ccccc6c(-c6ccc(-c7cc(-c8ccccc8)cc(-c8ccccc8)c7)cc6)c6ccccc56)c5ccccc45)cc3)c2)cc1]

7. The electroluminescent material prepared by and raw material according to claim 6, wherein the first compound is 9,9′-bianthryl derivative, and the second compound is 1-bromo-3,5-biphenyl, and the 9,9′-bianthryl derivative has a chemical formula as follows: 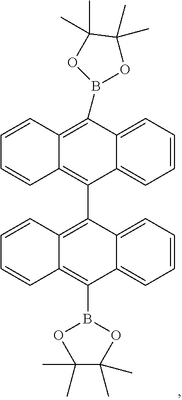 [SMILES: CC1(C)OB(c2c3ccccc3c(-c3c4ccccc4c(B4OC(C)(C)C(C)(C)O4)c4ccccc34)c3ccccc23)OC1(C)C]
and the 1-bromobenzene-3,5-biphenyl has a chemical formula as follows:

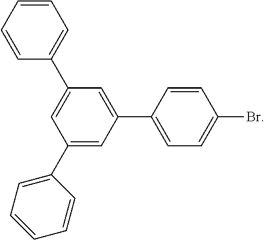 [SMILES: Brc1ccc(-c2cc(-c3ccccc3)cc(-c3ccccc3)c2)cc1]

8. The electroluminescent material prepared by the raw material according to claim 1, further comprising 10-triphenyl, 10′-tetraphenylenyl-9,9′-bianthryl derivative, and the 10-triphenyl, 10′-tetraphenylenyl-9,9′-bianthryl derivative has a chemical formula as follows:

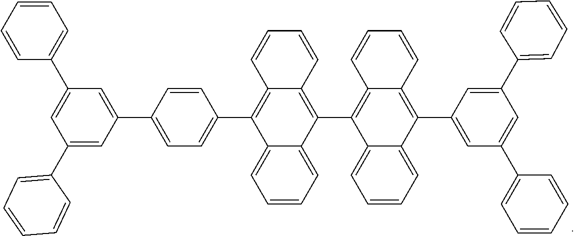 [SMILES: c1ccc(-c2cc(-c3ccccc3)cc(-c3ccc(-c4c5ccccc5c(-c5c6ccccc6c(-c6cc(-c7ccccc7)cc(-c7ccccc7)c6)c6ccccc56)c5ccccc45)cc3)c2)cc1]

9. The electroluminescent material prepared by the raw material according to claim 8, wherein the first compound is 9,9′-bianthryl derivative, and the second compound is a mixture of 1-bromo-3,5-biphenyl and 1-bromobenzene-3,5-biphenyl.

10. The electroluminescent material prepared by the raw material according to claim 9, wherein a ratio of a weight percentage of the 1-bromo-3,5-biphenyl to the 1-bromobenzene-3,5-biphenyl ranges from 0.8 to 1.2.
